(19) United States
(12) Patent Application Publication
(10) Pub. No.: US 2015/0005314 A1
(43) Pub. Date: Jan. 1, 2015

(54) PHARMACEUTICAL PREPARATION COMPRISING BREXPIRAZOLE AND SUBSTITUTED BETA-CYCLODEXTRIN

(71) Applicant: OTSUKA PHARMACEUTICAL CO., LTD., Tokyo (JP)

(72) Inventors: Tetsuya Hasegawa, Osaka (JP); Hidekazu Toyobuku, Osaka (JP)

(21) Appl. No.: 14/369,386

(22) PCT Filed: Dec. 28, 2012
(86) PCT No.: PCT/JP2012/084313

§ 371 Date: Jun. 27, 2014

Related U.S. Application Data
(60) Provisional application No. 61/580,708, filed on Dec. 28, 2011.

(51) Int. Cl. A61K 31/496; A61K 47/48

(52) U.S. Cl. CPC A61K 31/496; A61K 47/48969
USPC 514/253.07

(57) ABSTRACT

Provided is an aqueous pharmaceutical preparation comprising 7-[4-(4-benzo[b]thiophen-4-yl-piperazin-1-yl)butoxy]-1H-quinolin-2-one (compound (I)) or a salt thereof, which shows improved water solubility of compound (I) or a salt thereof achieved by addition of substituted β-cyclodextrin. The present invention provides a pharmaceutical preparation comprising compound (I) or a salt thereof, and substituted β-cyclodextrin.

### TECHNICAL FIELD

The present invention relates to a pharmaceutical preparation (pharmaceutical composition) comprising 7-[4-(4-benzo[b]thiophen-4-yl-piperazin-1-yl)butoxy]-1H-quinolin-2-one or a salt thereof and substituted β-cyclodextrin.

### BACKGROUND ART

It is known that 7-[4-(4-benzo[b]thiophen-4-yl-piperazin-1-yl)butoxy]-1H-quinolin-2-one (hereinafter to be referred to as compound (I)) or a salt thereof has dopamine D₂ receptor partial agonist action, serotonin 5-HT_{2A} receptor antagonist action and adrenaline α₁ receptor antagonist action, and further has a serotonin uptake inhibitory action (or serotonin reuptake inhibitory action) in addition to those actions (patent document 1), and has a wide treatment spectrum for central neurological diseases (particularly schizophrenia). However, since compound (I) and a salt thereof are poorly soluble in water, an aqueous pharmaceutical preparation thereof is difficult to produce.

Cyclodextrin has a function to form an inclusion complex with a hydrophobic molecule, and is known to provide an effect to increase the solubility of a particular drug. However, there are many drugs that are not capable of forming a complex with cyclodextrin, or fail to provide a clear advantage. For example, such drugs are disclosed in J. Szejtli,

Cyclodextrinsin Drug Formulations: Part II, Pharmaceutical Technology, 24-38, August, 1991 (non-patent document 1).

U.S. Pat. Nos. 5,134,127 (patent document 2) and 5,376,645 (patent document 3) disclose a sulfoalkyl ether cyclodextrin derivative and use of said derivative as a solubilizer of water-insoluble drugs for oral, intranasal or parenteral administration including intravenous and intramuscular administrations. In addition, they disclose an inclusion complex of water-insoluble drug and a sulfoalkyl ether cyclodextrin derivative and pharmaceutical compositions containing the complex. Examples of the disclosed sulfoalkyl ether cyclodextrin derivative include monosulfobutyl ether of β-cyclodextrin and monosulfopropyl ether of β-cyclodextrin. Examples of the water-insoluble drug include benzodiazepine, chlorpromazine, diazepam, mephobarbital, metharbital, nitrazepam and phenobarbital.

U.S. Pat. No. 6,232,304 (patent document 4) discloses an inclusion complex of a salt of an arylheterocyclo compound, which includes, for example, ziprasidone tartrate in cyclodextrin such as sulfobutyl ether (3-cyclodextrin (SBECD) and hydroxypropyl β-cyclodextrin (HPBCD), and also discloses use of such inclusion complexes for oral agents and parenteral agents.

U.S. Pat. No. 5,904,929 (patent document 5) discloses a pharmaceutical composition for transmucosal or transdermal administration, which contains a drug, and peracylated cyclodextrin as a solubilizer. Examples of the drug include antidepressants such as amitriptyline HCl, amoxapine, butriptyline HCl, clomipramine HCl, desipramine HCl, dothiepin HCl, doxepin HCl, fluoxetine, gepirone, imipramine, lithium carbonate, mianserin HCl, milnacipran, nortriptyline HCl and paroxetine HCl; anti-muscarinic agents such as atropine sulphate and hyoscine; sedating agents such as alprazolam, buspirone HCl, chlordiazepoxide HCl, chlorpromazine, clozapine, diazepam, flupenthixol HCl, fluphenazine, flurazepam, lorazepam, mazapertine, olanzapine, oxazepam, pimozide, pipamperone, piracetam, promazine, risperidone, selfotel, seroquel, sulpiride, temazepam, thiothixene, triazolam, trifluperidol and ziprasidone; anti-migraine drugs such as alniditan and sumatriptan; beta-adrenoreptor blocking agents such as atenolol, carvedilol, metoprolol, nebivolol and propranolol; anti-Parkinsonian drugs such as bromocryptine mesylate, levodopa and selegiline HCl; opioid analgesics such as buprenorphine HCl, codeine, dextromoramide and dihydrocodeine; parasympathomimetics such as galanthamine, neostigmine, physostymine, tacrine, donepezil, ENA 713 (exelon) and xanomeline; and vasodilators such as amlodipine, buflomedil, amyl nitrite, diltiazem, dipyridamole, glyceryl trinitrate, isosorbide dinitrate, lidoflazine, molsidomine, nicardipine, nifedipine, oxpentifylline and pentaerythritol tetranitrate.

JP-A-2006-501240 (patent document 6) discloses a preparation containing an inclusion complex of aripiprazole in sulfobutyl ether β-cyclodextrin (SBECD).

### DOCUMENT LIST

### [Patent Documents]

patent document 1: JP-A-2006-316052
patent document 2: U.S. Pat. No. 5,134,127
patent document 3: U.S. Pat. No. 5,376,645
patent document 4: U.S. Pat. No. 6,232,304
patent document 5: U.S. Pat. No. 5,904,929
patent document 6: JP-A-2006-501240

### [Non-Patent Document]

non-patent document 1: J. Szejtli, Cyclodextrinsin Drug Formulations: Part II, Pharmaceutical Technology, 24-38, August, 1991

### SUMMARY OF THE INVENTION

### Problems to be Solved by the Invention

The present invention aims to provide an aqueous pharmaceutical preparation comprising compound (I) or a salt thereof, by improving the water solubility of compound (I) or a salt thereof.

### Means of Solving the Problems

The present inventors have conducted various studies in an attempt to solve the above-mentioned problem, and found that the water solubility of compound (I) or a salt thereof is sufficiently improved by adding substituted β-cyclodextrin, and an aqueous pharmaceutical preparation (particularly, an aqueous preparation for injection) thereof can be produced.

In addition, the present inventors have found that compound (I) or a salt thereof forms an inclusion complex with substituted β-cyclodextrin, and the inclusion complex shows good water-solubility.

The present invention has been completed as a result of further studies based on the above-mentioned findings, and provides the following.

Accordingly, the present invention relates to the following [1]-[19].
[1] A pharmaceutical preparation comprising compound (I) or a salt thereof, and substituted β-cyclodextrin.
[2] The preparation of the above-mentioned [1], wherein the substituted β-cyclodextrin is sulfobutyl ether β-cyclodextrin or hydroxypropyl β-cyclodextrin.
[3] The preparation of the above-mentioned [1], wherein the substituted β-cyclodextrin is sulfobutyl ether β-cyclodextrin.
[4] The preparation of any of the above-mentioned [1]-[3], which is a preparation for injection.
[5] The preparation of any of the above-mentioned [1]-[4], is an aqueous preparation for injection.
[6] The preparation of the above-mentioned [5], which has a pH of 3.5-5.
[7] The preparation of the above-mentioned [6], further comprising an acid buffering agent.
[8] The preparation of the above-mentioned [7], wherein the acid buffering agent is phosphoric acid, hydrochloric acid, succinic acid, acetic acid, tartaric acid, lactic acid, citric acid, malic acid or glycolic acid.
[9] The preparation of the above-mentioned [8], wherein the acid buffering agent is tartaric acid.

[10] The preparation of any of the above-mentioned [1]-[9], wherein the weight ratio of the substituted β-cyclodextrin, and compound (I) or a salt thereof is 5:1-2000:1.
[11] The preparation of any of the above-mentioned [5]-[10], wherein the content of compound (I) or a salt thereof is 0.1-10 mg/mL.
[12] The preparation of any of the above-mentioned [1]-[11], wherein the substituted β-cyclodextrin is sulfobutyl ether β-cyclodextrin, and the weight ratio of sulfobutyl ether β-cyclodextrin, and compound (I) or a salt thereof is 10:1-1000:1.
[13] The preparation of any of the above-mentioned [1]-[12], wherein the compound (I) or a salt thereof and substituted β-cyclodextrin exist in the form of an inclusion complex.
[14] The preparation of the above-mentioned [13], wherein the amount of compound (I) or a salt thereof provided in the form of an inclusion complex, which is measured in an aqueous solution having a substituted β-cyclodextrin concentration of 150 mg/mL, is at least 0.2 mg/mL.

[15] An aqueous preparation for injection comprising compound (I) or a salt thereof, sulfobutyl ether β-cyclodextrin, tartaric acid, sodium hydroxide and water, and having pH within the range of about 4-4.6.
[16] The preparation of any of the above-mentioned [1]-[15], which is a preparation for muscle injection.
[17] An inclusion complex of substituted β-cyclodextrin and compound (I) or a salt thereof.
[18] The inclusion complex of the above-mentioned [17], wherein the substituted β-cyclodextrin is sulfobutyl ether β-cyclodextrin or hydroxypropyl β-cyclodextrin.
[19] The inclusion complex of the above-mentioned [18], wherein the substituted β-cyclodextrin is sulfobutyl ether β-cyclodextrin.

### Effect of the Invention

According to the present invention, the water solubility of compound (I) or a salt thereof can be sufficiently improved by adding substituted β-cyclodextrin, and an aqueous pharmaceutical preparation comprising compound (I) or a salt thereof can be provided.

### DESCRIPTION OF EMBODIMENTS

In the present invention, compound (I) or a salt thereof is contained as an active ingredient.

Compound (I) or a salt thereof can be produced according to the method described in the above-mentioned patent document 1, or a method analogous thereto.

While the salt of compound (I) usable in the present invention is not particularly limited as long as it is a pharmacologically acceptable salt, for example, inorganic acid salts such as sulfate, nitrate, hydrochloride, phosphate, hydrobromide and the like; organic acid salts such as acetate, sulfonates such as p-toluenesulfonate, methanesulfonate, ethanesulfonate and the like, oxalate, maleate, fumarate, malate, tartrate, citrate, succinate, benzoate and the like can be used.

The “substituted β-cyclodextrin” in the present invention includes, for example, a compound obtainable by modification of one or more hydroxyl groups of β-cyclodextrin, such as hydroxyalkylation (e.g., hydroxypropylation), sulfoalkyl etherification (e.g., sulfobutyl etherification), methylation, carboxymethylation, benzylation, polyethylene glycolation, aminoethylation and the like. Specifically, the “substituted β-cyclodextrin” in the present invention includes, for example, a compound wherein one or more hydroxyl groups of β-cyclodextrin are substituted by —O—CH₂—CH(OH)—CH₃, —O—(CH₂)₄—SO₃⁻ and the like.

For the purpose of the present invention, an average number of substituents to be introduced into substituted β-cyclodextrin is preferably 2-10, more preferably 4-9, per molecule.

The substituted β-cyclodextrin can be produced by a method known per se, and a commercially available product sold with a trade name of, for example, “2-hydroxypropyl-β-cyclodextrin” (manufactured by Wako Pure Chemical Industries, Ltd.), “Captisol” (manufactured by Cydex) and the like can also be used. In the present invention, one or more kinds selected from the aforementioned substituted β-cyclodextrins can be used.

As the substituted β-cyclodextrin to be used in the present invention, sulfoalkyl ether β-cyclodextrin and hydroxyalkyl β-cyclodextrin are preferable, sulfobutyl ether β-cyclodextrin (SBECD) and hydroxypropyl β-cyclodextrin (HPBCD) are more preferable, and SBECD is particularly preferable.

The pharmaceutical preparation of the present invention is provided in a preferable form of an aqueous parenteral preparation or a preparation for injection (particularly preparation for muscle injection). The pharmaceutical preparation of the present invention may also be in a dosage form of, for example, freeze-dry injection, oral preparation (e.g., tablet, capsule, elixir etc.), transdermal agent, transmucosal agent or inhalant and the like.

The preparation for injection in the present invention includes an aqueous preparation for injection and freeze-dry injection.

In the pharmaceutical preparation of the present invention (particularly aqueous preparation for injection), the weight ratio of the substituted β-cyclodextrin, and compound (I) or a salt thereof (substituted β-cyclodextrin: compound (I) or a salt thereof) is generally 5:1-2000:1, preferably 10:1-1000:1, more preferably 20:1-500:1.

The amount of the substituted β-cyclodextrin necessary for inhibiting or preventing precipitation of compound (I) or a salt thereof at an administration site varies depending on the kind of substituted β-cyclodextrin to be used.

For example, in the pharmaceutical preparation of the present invention (particularly aqueous preparation for injection), when the substituted β-cyclodextrin is SBECD, the weight ratio of SBECD, and compound (I) or a salt thereof (SBECD:compound (I) or a salt thereof) is preferably 10:1-1000:1, more preferably 20:1-500:1.

Since excess substituted β-cyclodextrin aids dissolution of compound (I) or a salt thereof, substituted β-cyclodextrin may be present in an amount more than necessary for forming an inclusion complex with compound (I) or a salt thereof in the pharmaceutical preparation of the present invention.

In the pharmaceutical preparation of the present invention, the content of compound (I) or a salt thereof varies depending on the dosage form and the like. For example, when it is an aqueous preparation for injection, the content is generally about 0.1- about 10 mg/mL, more preferably about 0.2- about 4 mg/mL.

The amount of the aqueous preparation for injection of the present invention to be filled in a container such as vial and the like is preferably 0.5-2 mL.

In the pharmaceutical preparation of the present invention, the content of the substituted β-cyclodextrin varies depending on the dosage form and the like. For example, when it is an aqueous preparation for injection, the content is generally about 25- about 250 mg/mL, preferably about 50-200 mg/mL, more preferably about 100- about 200 mg/mL.

When the pharmaceutical preparation of the present invention is an aqueous preparation for injection, the pH of said preparation is preferably about 3.5- about 5, more preferably about 4- about 4.6, further preferably about 4.3, from the aspect of solubility.

In the aqueous preparation for injection of the present invention, pH is preferably buffered within the above-mentioned range.

The method for adjusting or buffering the pH of an aqueous preparation for injection to fall within the above-mentioned range is not particularly limited, and a method known in the field of pharmaceutical preparation may be used. For example, a buffering agent containing an acid or a salt thereof is used.

Examples of the acid include phosphoric acid, hydrochloric acid, succinic acid, acetic acid, tartaric acid, lactic acid, citric acid, malic acid or glycolic acid and the like. Of these, tartaric acid, citric acid, lactic acid, phosphoric acid and hydrochloric acid are preferable, and tartaric acid is most preferable.

Where necessary, pH may be adjusted to fall within the above-mentioned range by adding a base such as hydroxide of alkali metal (e.g., sodium hydroxide, potassium hydroxide or lithium hydroxide, preferably sodium hydroxide); or hydroxide of alkaline earth metal (e.g., magnesium hydroxide or calcium hydroxide) and the like.

As the aqueous preparation for injection of the present invention, an aqueous preparation for injection comprising compound (I) or a salt thereof, SBECD, tartaric acid, sodium hydroxide and water, and having pH within the range of about 4-4.6 is preferable.

Moreover, as the aqueous preparation for injection of the present invention, a preparation comprising the following components is preferable.
(1) about 0.2- about 4 mg/mL of compound (I) or a salt thereof
(2) about 100- about 200 mg/mL of SBECD
(3) about 7-9 mg/mL of an acid (preferably tartaric acid) or a salt thereof for adjusting pH to the range of about 3.5- about 5
(4) a base (preferably alkali metal hydroxide, preferably sodium hydroxide) for further adjusting pH to the range of about 4- about 4.6 and
(5) water to make the total volume 1 mL.

The pharmaceutical preparation of the present invention can comprise a general additive used for general formulation as long as the characteristics of the present invention are not impaired. Examples of such additive include excipient, emulsifier, suspending agent, preservative, corrigent, film coating agent, colorant, flavoring agent and the like. Particularly, for an aqueous preparation for injection, other solubilizing agents such as sorbitol, propylene glycol, polyoxyethylene sorbitan monolaurate and the like; isotonicity agents such as potassium chloride, sodium chloride, glycerol and the like; stabilizers such as sodium edetate and the like; antioxidants such as ascorbic acid and the like; soothing agents such as meprylcaine hydrochloride, lidocaine hydrochloride, etc. and the like can be recited as examples.

The pharmaceutical preparation of the present invention can be produced by a conventional method, for example, the method described in preparation General Rules of the Japanese Pharmacopoeia, US Pharmacopeia, etc. and the like.

The dosage form of an aqueous preparation for injection can be produced by, though not particularly limited to, a method including, for example, dissolving by adding compound (I) or a salt thereof, and substituted β-cyclodextrin together with a buffering agent such as an acid or a salt thereof and the like, and other additives to water for injection that meets the standards of, for example, the Japanese Pharmacopoeia, US Pharmacopeia and the like, filling the homogenized solution in a container, tightly sealing and sterilizing the same; or by dissolving by adding the aforementioned components to water for injection, and aseptically filtering the homogenized solution or aseptically preparing to give a homogenized solution, and filling the solution in a container and tightly sealing the same.

The aqueous preparation for injection of the present invention can be specifically prepared, for example, as follows.

An acid such as tartaric acid and the like or a salt thereof is dissolved in water for injection. Substituted β-cyclodextrin (preferably SBECD) is dissolved in the obtained aqueous solution, and then compound (I) or a salt thereof is dissolved. Then, a base such as sodium hydroxide, other alkali metal hydroxide or alkaline earth metal hydroxide and the like is added, and pH of said solution is adjusted to about 3.5-35 about 5, preferably about 4- about 4.6, more preferably about 4.3, and water is added to give a desired volume.

The obtained solution is aseptically filtered through, for example, a 0.22 μm-membrane filter, and filled in a vial. The vial is tightly sealed and finally sterilized.

In the aqueous preparation for injection of the present invention, generally, compound (I) or a salt thereof and substituted β-cyclodextrin form an inclusion complex wherein compound (I) or a salt thereof is a guest molecule and substituted β-cyclodextrin is a host molecule.

Not only a pharmaceutical preparation comprising compound (I) or a salt thereof, and substituted β-cyclodextrin as an inclusion complex, but also a pharmaceutical preparation comprising a physical mixture thereof are similarly encompassed in the present invention.

Such inclusion complex or physical mixture thereof is added to various pharmaceutically acceptable carriers such as liquid, emulsion, gel, powder and the like to give a pharmaceutical preparation, which can be provided in various dosage forms such as liquid, emulsion, gel, powder, granule, pill, tablet, capsule, aerosol and the like.

In the present invention, the inclusion complex of compound (I) or a salt thereof and substituted β-cyclodextrin may be formed in advance and added to the above-mentioned carrier, or each of compound (I) or a salt thereof, and substituted β-cyclodextrin may be separately added to the above-mentioned carrier and mixed or administered to allow them to form a complex in a solution, or may be formed in vivo (in gastrointestinal tract or oral cavity).

The pharmaceutical preparation of the present invention may be formulated as a physically dried mixture of compound (I) or a salt thereof and substituted β-cyclodextrin, or a dried inclusion complex thereof, and may be reconstituted as a preparation for injection by adding water. As a different method, an aqueous preparation for injection may be freeze-dried and thereafter reconstituted as a preparation for injection by adding water.

When compound (I) or a salt thereof and substituted β-cyclodextrin contained in the pharmaceutical preparation of the present invention are contained in the form of an inclusion complex and the concentration of substituted β-cyclodextrin is 150 mg/mL, the amount of compound (I) or a salt thereof in said complex is preferably at least 0.2 mg/mL, more preferably 4 mg/mL or less.

The pharmaceutical preparation of the present invention preferably in the form of an aqueous preparation for injection can be used for the treatment of schizophrenia and associated disorders (e.g., bipolar disorder and dementia) and the like in human patients. In the aqueous preparation for injection of the present invention, a preferable dose of compound (I) or a salt thereof is 0.05-6 mg per day for an adult. The aqueous preparation for injection of the present invention is preferably administered intramuscularly, but is also effective by subcutaneous injection or intravenous injection.

Thus, the present invention also provides a method of treating schizophrenia and associated disorders, comprising administering the above-mentioned aqueous preparation for injection preferably intramuscularly to patients in need of the treatment.

In the aqueous preparation for injection of the present invention, water solubility of compound (I) or a salt thereof is improved, and precipitation upon administration is suppressed. Therefore, the preparation is preferably administered intramuscularly for a good treatment of schizophrenia and associated disorders.

The present invention also provides an inclusion complex of substituted β-cyclodextrin and compound (I) or a salt thereof. The “substituted β-cyclodextrin” and “compound (I) or a salt thereof” are as explained for the above-mentioned pharmaceutical preparation of the present invention.

### EXAMPLES

The present invention is explained in more detail in the following by referring to Examples, which are not to be construed as limitative.

In the Examples, 7-[4-(4-benzo[b]thiophen-4-yl-piperazin-1-yl)butoxy]-1H-quinolin-2-one is compound (I).

A colorless transparent aqueous preparation for injection essentially having no problem by visual inspection (compound (I) 4 mg/mL, 8 mg/vial) was prepared as follows;

An adequate amount of water for injection was filled in a stainless reaction vessel, and tartaric acid granules (8.58 g) and sulfobutyl ether β-cyclodextrin (SBECD, 165 g) were added to the reaction vessel and dissolved in the stirring water.

Compound (I) (4.4 g) was added to the reaction vessel, and dissolved by stirring.

A 1N aqueous sodium hydroxide solution was added to the above-mentioned solution to adjust the pH to about 4.3.

Water for injection was added to the above-mentioned solution to the final volume of 1.1 L with stirring.

The above-mentioned solution was aseptically filtered through a 0.22 μm-membrane filter and filled in an aseptic container. The above-mentioned solution (8 mg as compound (I)) was filled in an aseptic vial and the vial was tightly sealed aseptically.

### INDUSTRIAL APPLICABILITY

According to the present invention, water solubility of compound (I) or a salt thereof is sufficiently improved by adding substituted β-cyclodextrin, and an aqueous pharmaceutical preparation comprising compound (I) or a salt thereof can be provided.

The present application is based on U.S. provisional application No. 61/580,708, the contents of which are encompassed in full herein.

1. A pharmaceutical composition comprising 7-[4-(4-benzo[b]thiophen-4-yl-piperazin-1-yl)butoxy]-1H-quinolin-2-one or a salt thereof, and substituted β-cyclodextrin.

2. The composition according to claim 1, wherein the substituted β-cyclodextrin is selected the group 1 sulfobutyl ether β-cyclodextrin and hydroxypropyl β-cyclodextrin.

3. The composition according to claim 2, wherein the substituted β-cyclodextrin is sulfobutyl ether β-cyclodextrin.
